(19) United States
(12) Patent Application Publication
(10) Pub. No.: US 2023/0181568 A1
(43) Pub. Date: Jun. 15, 2023

(54) Novel pharmaceutical compositions for emergency treatment of life-threatening opioid overdose and methods for fabrication and using thereof

(71) Applicant: Garry Alan Katz, Brighton, MI (US)

(72) Inventor: Garry Alan Katz, Brighton, MI (US)

(21) Appl. No.: 17/644,078

(22) Filed: Dec. 13, 2021

(51) Int. Cl. A61K 31/485

(52) U.S. Cl. CPC A61K 31/485

(57) ABSTRACT

This invention discloses methods of preparation and usage of novel pharmaceutical compositions for the emergency treatment of life-threatening opioid overdose. The compositions targets the opiate receptors with two different opioid antagonists. A quick onset, short acting opioid antagonist and a slower onset, longer acting opioid antagonist.

### CROSS-REFERENCE

Not applicable.

### FIELD OF INVENTION

The invention pertains to the field of pharmaceutical science and emergency medicine.

### BACKGROUND OF INVENTION

The United States is experiencing a record number of opioid overdoses and deaths.

In 1971 naloxone was approved by the FDA as an antidote for opioid overdose. Naloxone is a competitive opioid antagonist and is the drug of choice for emergency opioid overdose. It is available for IV, SC, IM and intranasal administration.

Laws have been passed in all 50 states and the district of Colombia to increase the public access to naloxone. EMT's, police and family members have access to naloxone and have been trained to administer it.

The problem with the use of naloxone by itself is that most opiates duration of action exceeds the duration of action of naloxone and may wear off in as little as a few minutes, requiring repeated doses of naloxone which might not be immediately available.

There exists a need for novel approach for treating life-threatening opioid overdoses in a more predictable manner. This patent allows for a more effective and safer treatment by the addition of a longer acting opioid blocker, naltrexone or nalmefene, decreasing the need for repeated dosing.

### DETAILED DESCRIPTION

The invention is a pharmaceutical composition for the emergency treatment of life-threatening opioid overdose. It consists of two different opioid antagonists, naloxone, a quick acting and short duration of action opiate antagonist and a slower acting and longer duration action opiate antagonist, naltrexone or nalmefene. Adding naltrexone or nalmefene decrease the need for repeated dosing. The opiate blockers can be given as a combination product or as individual agents.

The following are examples of preparing and use of invention.

### Example 1

Consists of 2 separate vials that are reconstituted immediately before intramuscular or subcutaneous injection.

| | |
|---|---|
| Vial #1 | |
| naloxone hydrochloride | 1 mg |
| naltrexone hydrochloride | 1.5 mg |
| sodium chloride | Added to adjust tonicity |
| Vial #2 | |
| Water for injection | 0.5 ml |
| hydrochloric acid | to adjust pH (pH range is 3.0 to 4.5) |

Method of preparation: Weigh and measure each ingredient.
Sterilize both vials.

Method of use: With syringe and needle inject vial #2 into vial #1 and shake until dissolved.
Draw up product with syringe and needle.
Administer dose intramuscularly or subcutaneous for the emergency treatment of opioid overdose.

### Example 2

Consist of an aqueous solution of naloxone hydrochloride and naltrexone hydrochloride combined in a pre-filled, multi-dose intranasal spray device.

| | |
|---|---|
| naloxone hydrochloride | 8 mg |
| naltrexone hydrochloride | 8 mg |
| purified water | 0.1 ml |
| sodium chloride | to adjust tonicity |
| hydrochloric acid | to adjust pH (pH range is 3.5 to 5.5) |
| benzalkonium chlorde | preservative |
| disodium ethylene- | stabilizer |
| diaminetetraacetate | |

Method of preparation of one dose: Weigh and measure each ingredient.
Combine and shake until dissolved.
Place 0.5 ml of combined ingredients in intranasal spray device that delivers 0.1 ml/spray.
Sterilize.

Method of use: Administer intranasal dose of 1 spray to each nostril for the emergency treatment of opioid overdose.

### Example 3

Consists of a kit containing two prefilled, ready to use , intranasal spray devices.

Device #1 contains naloxone hydrochloride and device #2 contains naltrexone hydrochloride.

| | |
|---|---|
| Device #1 | |
| naloxone hydrochloride | 8 mg |
| purified water | 0.1 ml |
| sodium chloride | to adjust tonicity |
| hydrochloric acid | to adjust pH (pH range is 3.5 to 5.5) |
| benalkonium chloride | preservative |
| disodium ethylene- | stabilizer |
| diaminetetraacetate | |

Method of preparation: Weigh or measure each ingredient.
Dissolve ingredients in purified water.
Adjust pH with hydrochloric acid.
Place combined ingredients in a single nasal spray device.

| | |
|---|---|
| Device #2 | |
| naltrexone hydrochloride | 8 mg |
| purified water | 0.1 ml |
| sodium chloride | to adjust tonicity |
| hydrochloric acid | to adjust pH (pH range is 3.5 to 5.5) |
| benzalkonium chloride | preservative |
| disodium ethylene- | stabilizer |
| diaminetetraacetate | |

Method of preparation: Weigh or measure each ingredient.
Dissolve ingredients in purified water.
Adjust pH with hydrochloric acid.
Place combined ingredients in a single nasal spray device.

Method of use: Administer intranasal dose of one spray from device #1 to one side and one spray from device #2 to the other side for the emergency treatment of opioid overdose.

### Example 4

Consists of a ready to use IM/SQ auto-injector containing naloxone and nalmefene.

| | |
|---|---|
| naloxone hydrochloride | 1 mg |
| nalmefene hydrochloride | 2 mg |
| purified water | 0.5 ml |
| sodium chloride | to adjust tonicity |
| hydrochloric acid | to adjust pH |

Method of preparation: Weigh or measure each ingredient.
Combine and dissolve ingredients.
Place combined ingredients in IM auto-injector.
Sterilize.

Method of use: Administer dose intramuscularly or subcutaneously for the emergency treatment of opioid overdose.

### SUMMARY

The present invention is a novel pharmaceutical composition for the emergency treatment of life-threatening opioid overdose.

The problem with the present treatment of opioid overdose, is that most opioids duration of action exceeds the duration of action of naloxone and may wear off and require repeated doses of naloxone which might not be available.

This novel pharmaceutical composition uses two different opiate blockers, a quick onset opiate blocker, naloxone, and a long acting opiate blocker, naltrexone or nalmefene.

This patent allows for a more effective and safer treatment of opioid overdose by decreasing the need for repeated dosing.

1. A pharmaceutical formulation for the treatment of opioid overdose consisting of two opioid antagonists, a fast acting and short duration of action opioid antagonist, naloxone , and a slower acting and longer duration of action opioid antagonist, naltrexone or nalmefene.

2. The pharmaceutical composition of claim 1, wherein the composition of the fast acting and short duration of action opioid antagonist is naloxone and pharmaceutically suitable salts or hydrate.

3. The pharmaceutical composition of claim 1, wherein the composition of the slower acting and longer duration of action drug is selected from group consisting of naltrexone and nalmefene and pharmaceutical suitable salts or hydrates.

4. The pharmaceutical composition of claim 1, wherein the dosage form is a IV/IM/SQ injection, topical dosage form, nasal liquid spray, nasal powder, nasal gel, rectal dosage form or oral dosage.

5. The pharmaceutical composition of claim 1, wherein the dosage of naloxone are between 0.1 ug to 100mg/dose.

6. The pharmaceutical composition of claim 1, wherein the dosage of naltrexone and nalmefene are between 0.1 ug to 100mg/dose.

7. The pharmaceutical composition of claim 1, wherein the opioid antagonist are given as a combination product or given individually, naloxone given first followed by naltrexone or nalmefene.
